(19) United States
(12) Patent Application Publication
(10) Pub. No.: US 2005/0244837 A1
(43) Pub. Date: Nov. 3, 2005

(54) Method and device for sample preparation control

(75) Inventor: William McMillan, Cupertino, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cepheid, Sunnyvale, CA (US)

(21) Appl. No.: 10/834,538

(22) Filed: Apr. 28, 2004

(51) Int. Cl. C12Q 1/68; C12N 1/08

(52) U.S. Cl. 435/6; 435/270

(57) ABSTRACT

A method for preparing a sample suspected to contain a target nucleic acid sequence for a nucleic acid amplification reaction and for verifying the effectiveness of the sample preparation comprises the step of mixing the sample with sample preparation controls. The sample preparation controls are cells, spores, microorganisms, or viruses that contain a marker nucleic acid sequence. The sample mixed with the sample preparation controls is subjected to a lysis treatment, and nucleic acid released by the lysis treatment is subjected to nucleic acid amplification conditions. The presence or absence of the target nucleic acid sequence and of the marker nucleic acid sequence is then determined. Positive detection of the marker nucleic acid sequence indicates that the sample preparation process was satisfactory, while the inability to detect the marker nucleic acid sequence indicates inadequate sample preparation.

### BACKGROUND OF THE INVENTION

The present invention relates generally to nucleic acid assays and, more particularly, to a device and method for preparing a sample for nucleic acid amplification and for verifying the integrity of the sample preparation process.

Methods for amplifying nucleic acids provide useful tools for the detection of human pathogens, detection of human genetic polymorphisms, detection of RNA and DNA sequences, for molecular cloning, sequencing of nucleic acids, and the like. In particular, the polymerase chain reaction (PCR) has become an important tool in the cloning of DNA sequences, forensics, paternity testing, pathogen identification, disease diagnosis, and other useful methods where the amplification of a nucleic acid sequence is desired. See e.g., PCR Technology: Principles and Applications for DNA Amplification (Erlich, ed., 1992); PCR Protocols: A Guide to Methods and Applications (Innis et al., eds, 1990).

The analysis of samples suspected of containing a nucleic acid sequence of interest generally involves a series of sample preparation steps, which may include filtration, cell lysis, nucleic acid purification, and mixing with reagents. To be confident about the results of a nucleic acid assay, it would be useful to control for the integrity of the sample preparation process. The present invention addresses this and other problems.

### SUMMARY

According to one aspect, the invention provides a method for preparing a sample for a nucleic acid amplification reaction and for verifying the effectiveness of the sample preparation. The sample is suspected of containing target entities selected from the group consisting of cells, spores, microorganisms, and viruses, and the target entities comprise at least one target nucleic acid sequence. The method comprises the step of introducing the sample into a device having a mixing chamber for mixing the sample with sample preparation controls. The sample preparation controls are selected from the group consisting of cells, spores, microorganisms, and viruses, and the sample preparation controls comprise a marker nucleic acid sequence. The device further has a lysing chamber and a reaction chamber. The sample is mixed with the sample preparation controls in the mixing chamber. The method further comprises the steps of subjecting the sample preparation controls and the target entities, if present in the sample, to a lysis treatment in the lysing chamber, subjecting nucleic acid released in the lysing chamber to nucleic acid amplification conditions in the reaction chamber, and detecting the presence or absence of the at least one target nucleic acid sequence and of the marker nucleic acid sequence. Positive detection of the marker nucleic acid sequence indicates that the sample preparation process was satisfactory, while the inability to detect the marker nucleic acid sequence indicates inadequate sample preparation.

In some embodiments, the lysing chamber contains solid phase material, and the method further comprises the step of forcing the sample mixed with the sample preparation controls to flow through the lysing chamber to capture the sample preparation controls and the target entities, if present in the sample, with the solid phase material prior to the lysis treatment. In some embodiments, the solid phase material comprises at least one filter having a pore size sufficient to capture the sample preparation controls and the target entities. The sample may be pre-filtered (e.g., to remove coarse material) prior to mixing the sample with the sample preparation controls. In some embodiments, the lysis treatment comprises subjecting the sample preparation controls and the target entities to ultrasonic energy using an ultrasonic transducer coupled to a wall of the lysing chamber. The lysis treatment may optionally comprise agitating beads in the lysing chamber. In some embodiments, the sample preparation controls are spores. In some embodiments, the mixing step comprises dissolving a dried bead containing the sample preparation controls. In some embodiments, the lysis treatment comprises contact with a chemical lysis agent. In some embodiments, the nucleic acid amplification conditions comprise polymerase chain reaction (PCR) conditions. In some embodiments, the presence or absence of the marker nucleic acid sequence is detected by determining if a signal from a probe capable of binding to the marker nucleic acid sequence exceeds a threshold level.

According to another aspect, the invention provides a device for preparing a sample for a nucleic acid amplification reaction and for verifying the effectiveness of the sample preparation. The sample is suspected of containing target entities selected from the group consisting of cells, spores, microorganisms, and viruses, and the target entities comprise at least one target nucleic acid sequence. The device comprises a body having a first chamber containing sample preparation controls to be mixed with the sample. The sample preparation controls are selected from the group consisting of cells, spores, microorganisms, and viruses, and the sample preparation controls comprise a marker nucleic acid sequence. The body also has a lysing chamber for subjecting the sample preparation controls and the target entities, if present in the sample, to a lysis treatment to release the nucleic acid therefrom. The body further has a reaction chamber for holding the nucleic acid for amplification and detection. The device further comprises at least one flow controller for directing the sample mixed with the sample preparation controls to flow from the first chamber into the lysing chamber and for directing the nucleic acid released in the lysing chamber to flow into the reaction chamber. The device further contains primers and probes for amplifying and detecting the marker nucleic acid sequence and the at least one target nucleic acid sequence.

In some embodiments, the lysing chamber contains solid phase material for capturing the sample preparation controls and the target entities, if present in the sample, as the sample flows through the lysing chamber, the device further includes at least one waste chamber for receiving used sample fluid that has flowed through the lysing chamber, and the at least one flow controller is further capable of directing used sample fluid that has flowed through the lysing chamber to flow into the waste chamber. In some embodiments, the solid phase material comprises at least one filter having a pore size sufficient to capture the sample preparation controls and the target entities. In some embodiments, the device further comprises an ultrasonic transducer coupled to a wall of the lysing chamber to sonicate the lysing chamber. In some embodiments, the device further comprises beads in the lysing chamber for rupturing the sample preparation controls and the target entities. In some embodiments, the sample preparation controls are spores. In some embodiments, the sample preparation controls are in a dried bead that is dissolvable in liquid. In some embodiments, the primers and probes are in a dried bead in the reaction chamber, the bead being dissolvable in liquid. In some embodiments, the body includes a mixing chamber connected to the reaction chamber, and the primers and probes are in a dried bead in the mixing chamber, the bead being dissolvable in liquid.

According to another aspect, the present invention provides a method for determining the effectiveness of a lysis procedure. The method comprises the steps of mixing sample preparation controls with a sample suspected of containing target entities selected from the group consisting of cells, spores, microorganisms, and viruses. The target entities comprise at least one target nucleic acid sequence. The sample preparation controls are selected from the group consisting of cells, spores, microorganisms, and viruses, and the sample preparation controls comprise a marker nucleic acid sequence. The mixture of the sample preparation controls and the target entities, if present in the sample, are subjected to a lysis treatment. The method further comprises the steps of detecting the presence or absence of the marker nucleic acid sequence to determine if nucleic acid was released from the sample preparation controls during the lysis treatment. Positive detection of the marker nucleic acid sequence indicates satisfactory lysis, while the inability to detect the marker nucleic acid sequence indicates inadequate lysis.

In some embodiments, the method further comprises the step of forcing the sample mixed with the sample preparation controls to flow through a chamber containing solid phase material to capture the sample preparation controls and the target entities, if present in the sample, with the solid phase material prior to the lysis treatment. In some embodiments, the solid phase material comprises at least one filter having a pore size sufficient to capture the sample preparation controls and the target entities. In some embodiments, the sample is pre-filtered prior to mixing the sample with the sample preparation controls. In some embodiments, the lysis treatment comprises subjecting the sample preparation controls and the target entities to ultrasonic energy. The lysis treatment may also comprise agitating beads to rupture the sample preparation controls and the target entities. In some embodiments, the sample preparation controls are spores. In some embodiments, the mixing step comprises dissolving a dried bead containing the sample preparation controls. In some embodiments, the lysis treatment comprises contact with a chemical lysis agent. In some embodiments, the marker nucleic acid sequence is detected by amplifying the marker nucleic acid sequence (e.g., by PCR) and detecting the amplified marker nucleic acid sequence. In some embodiments, the amplified marker nucleic acid sequence is detected by determining if a signal from a probe capable of binding to the marker nucleic acid sequence exceeds a threshold level.

### BRIEF DESCRIPTION OF THE DRAWINGS

FIG. 1 is a perspective view of the fluid control and processing device according to an embodiment of the present invention;

FIG. 2 is another perspective view of the device of FIG. 1;

FIG. 3 is an exploded view of the device of FIG. 1;

FIG. 4 is an exploded view of the device of FIG. 2;

FIG. 5 is an elevational view of a fluid control apparatus and gasket in the device of FIG. 1;

FIG. 6 is a bottom plan view of the fluid control apparatus and gasket of FIG. 5;

FIG. 7 is a top plan view of the fluid control apparatus and gasket of FIG. 5;

FIG. 8 is a cross-sectional view of the rotary fluid control apparatus of FIG. 7 along 8-8;

FIGS. 9A-9LL are top plan views and cross-sectional views illustrating a specific protocol for controlling and processing fluid using the fluid control and processing device of FIG. 1;

FIG. 10 is a cross-sectional view of a piston assembly; and

FIG. 11 is a cross-sectional view of a side-filtering chamber.

### DESCRIPTION OF THE SPECIFIC EMBODIMENTS

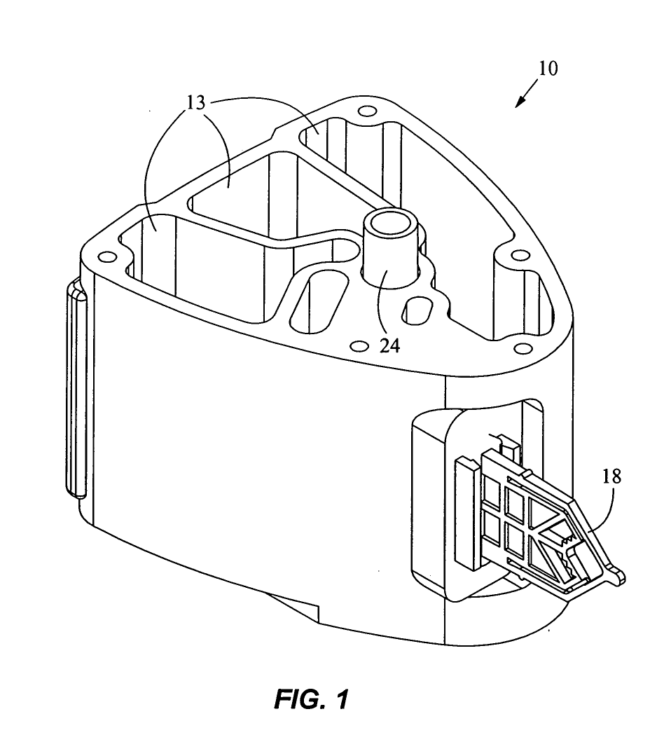

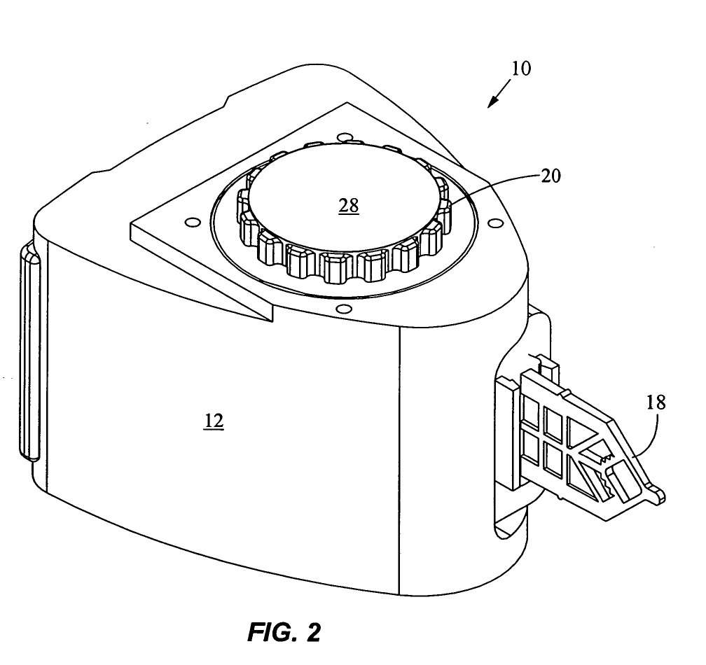

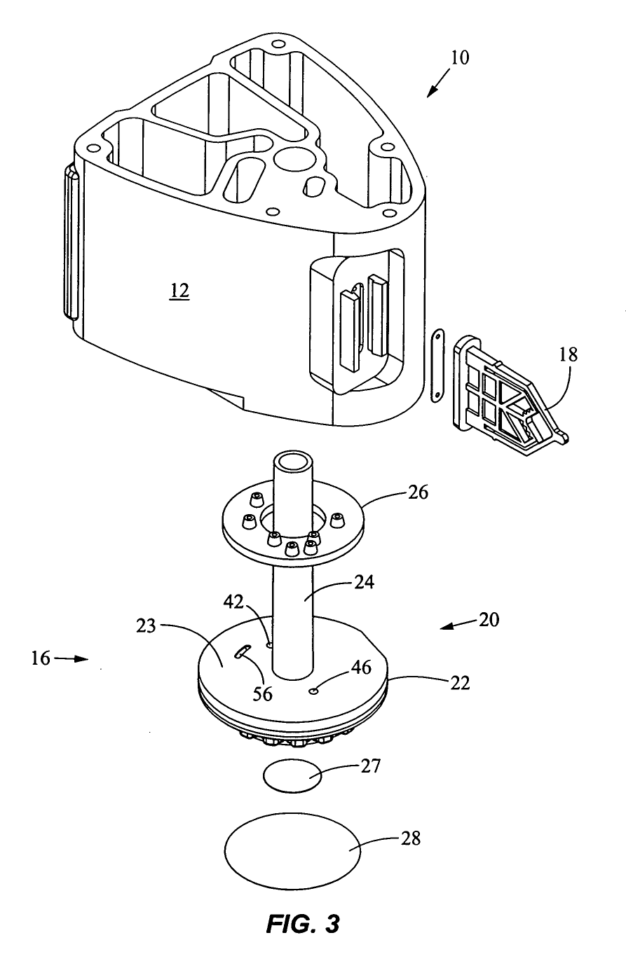

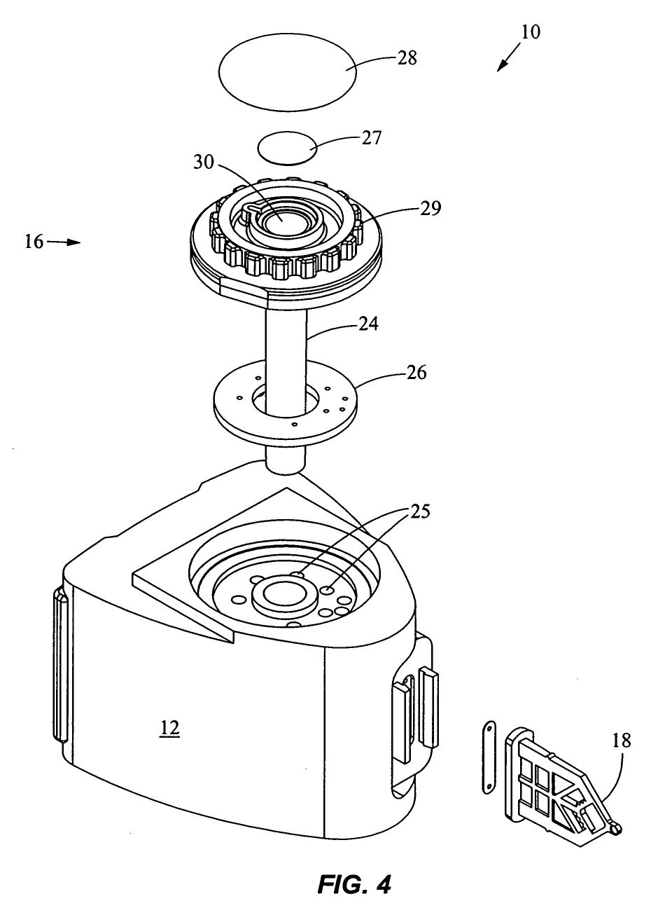

FIGS. 1-4 show a fluid control and processing system 10 including a housing 12 having a plurality of chambers 13. FIG. 1 shows the chambers 13 exposed for illustrative purposes. A top cover will typically be provided to enclose the chambers 13. As best seen in FIGS. 3 and 4, a fluid control device 16 and a reaction vessel 18 are connected to different portions of the housing 12. The fluid control device in the embodiment shown is a rotary fluid control valve 16. The valve 16 includes a valve body 20 having a disk portion 22 and a tubular portion 24. The disk portion 22 has a generally planar external port surface 23, as best seen in FIG. 3. The valve 16 is rotatable relative to the housing 12. The housing 12 includes a plurality of chamber ports 25 facing the external port surface 23 of the disk portion 22 of the valve 16 (FIG. 4) to permit fluidic communication between the chambers 13 and the valve 16. An optional seal or gasket 26 is disposed between the disk portion 22 and the housing 12. The disk portion 22 further includes a filter 27 and an outer wall 28, and a toothed periphery 29.

As seen in FIG. 4, the disk portion 22 includes a lysing chamber 30. The lysing chamber 30 may contain solid phase material for capturing cells, spores, viruses, or microorganisms to be lysed. Suitable solid phase materials include, without limitation, filters, beads, fibers, membranes, filter paper, glass wool, polymers, or gels. In a specific embodiment, the solid phase material is a filter having a pore size sufficient to capture target cells, spores, viruses, or microorganisms to be lysed.

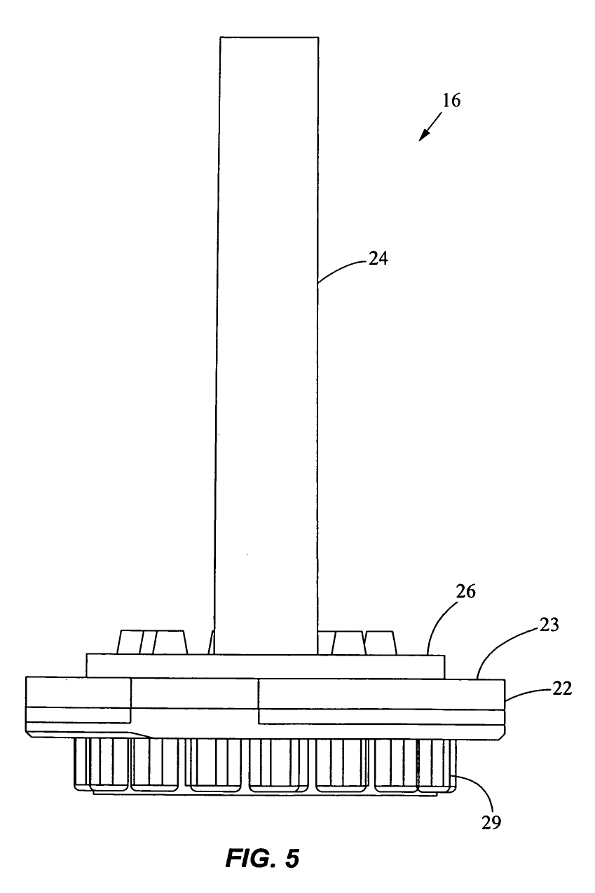

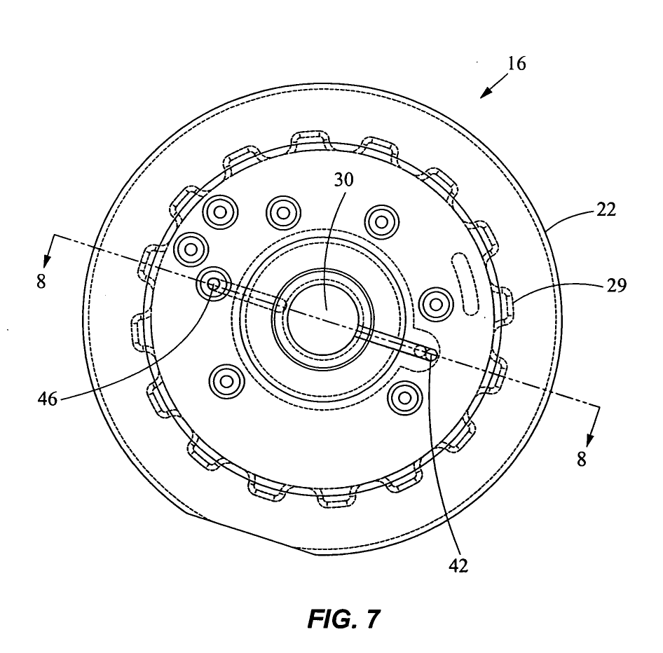

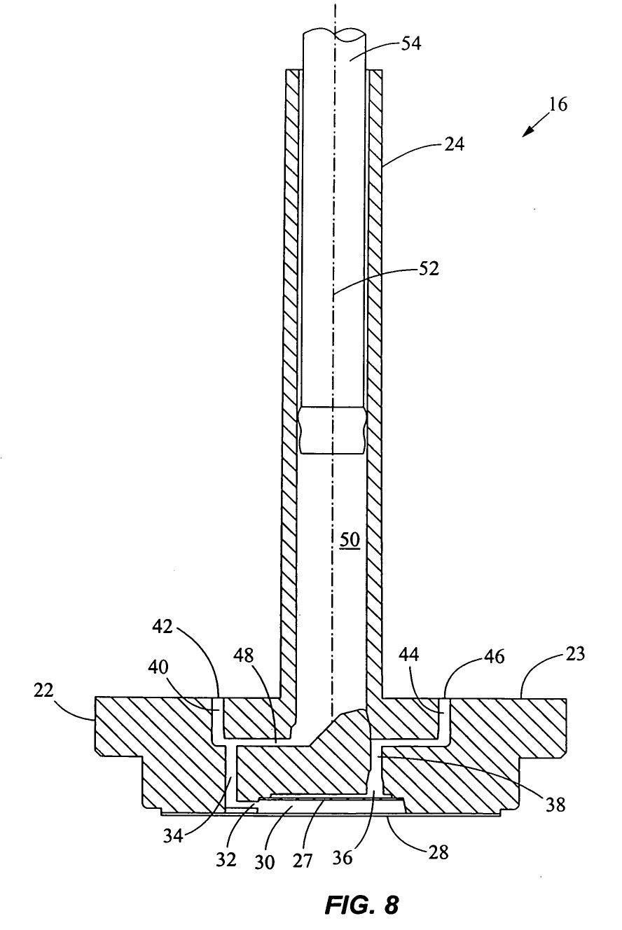

As shown in FIGS. 5-8, the outer wall 28 encloses the lysing chamber 30 and the bottom end of the disk portion 22 of the valve 16. In FIG. 8, the lysing chamber 30 includes a first fluid processing port 32 coupled to a first fluid processing channel 34, and a second fluid processing port 36 coupled to a second fluid processing channel 38. The first fluid processing channel 34 is coupled to a first outer conduit 40 ending at a first external port 42 at the external port surface 23, while the second fluid processing channel 38 is coupled to a second outer conduit 44 ending at a second external port 46 at the external port surface 23. A fluid displacement channel 48 is coupled to the first fluid processing channel 34 and first conduit 40 near one end, and to a fluid displacement chamber 50 at the other end. The first outer conduit 40 serves as a common conduit for allowing fluidic communication between the first external port 42 and either or both of the first fluid processing channel 34 and the fluid displacement channel 48. The lysing chamber 30 is in continuous fluidic communication with the fluid displacement chamber 50.

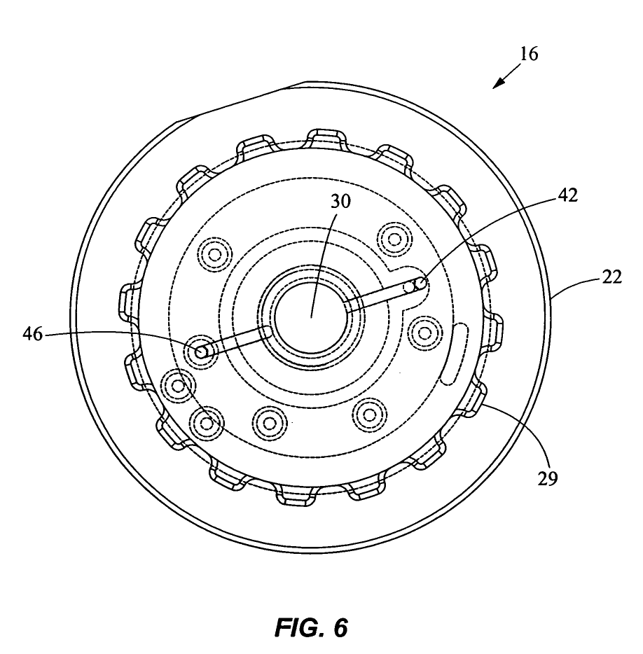

As shown in FIGS. 6-8, the external ports 42, 46 are angularly spaced from one another relative to the axis 52 of the valve 16 by about 180°. The external ports 42, 46 are spaced radially by the same distance from the axis 52. The axis 52 is perpendicular to the external port surface 23. In another embodiment, the angular spacing between the external ports 42, 46 may be different. The configuration of the channels in the disk portion 22 may also be different in another embodiment. For example, the first fluid processing channel 34 and the first outer conduit 40 may be slanted and coupled directly with the fluid displacement chamber 50, thereby eliminating the fluid displacement channel 48. The second fluid displacement channel 38 may also be slanted and extend between the second fluid processing port 36 and the second external port 46 via a straight line, thereby eliminating the second outer conduit 44. In addition, more channels and external ports may be provided in the valve 16. As best seen in FIG. 3, a crossover channel or groove 56 is desirably provided on the external port surface 23. The groove 56 is curved and desirably is spaced from the axis 52 by a constant radius. In one embodiment, the groove 56 is a circular arc lying on a common radius from the axis 52. As discussed in more detail below, the groove 56 is used for filling the vessel.

As shown in FIG. 8, the fluid displacement chamber 50 is disposed substantially within the tubular portion 24 of the valve 16 and extends partially into the disk portion 22. A fluid displacement member in the form of a plunger or piston 54 is movably disposed in the chamber 50. When the piston 54 moves upward, it expands the volume of the chamber 50 to produce suction for drawing fluid into the chamber 50. When the piston 54 moves downward, it decreases the volume of the chamber 50 to drive fluid out of the chamber 50.

As the rotary valve 16 is rotated around its axis 52 relative to the housing 12 of FIGS. 1-4, one of the external ports 42, 46 may be open and fluidicly coupled with one of the chambers 13 or reaction vessel 18, or both external ports 42, 46 may be blocked or closed. In this embodiment, at most only one of the external ports 42, 46 is fluidicly coupled with one of the chambers or reaction vessel 18. Other embodiments may be configured to permit both external ports 42, 46 to be fluidicly coupled with separate chambers or the reaction vessel 18. Thus, the valve 16 is rotatable with respect to the housing 12 to allow the external ports 42, 46 to be placed selectively in fluidic communication with a plurality of chambers which include the chambers 13 and the reaction vessel 18. Depending on which external port 42, 46 is opened or closed and whether the piston 54 is moved upward or downward, the fluid flow in the valve 16 can change directions, the external ports 42, 46 can each switch from being an inlet port to an outlet port, and the fluid flow may pass through the processing region 30 or bypass the lysing chamber 30. In a specific embodiment, the first external port 42 is the inlet port so that the inlet side of the lysing chamber 30 is closer to the fluid displacement chamber 54 than the outlet side of the lysing chamber 30.

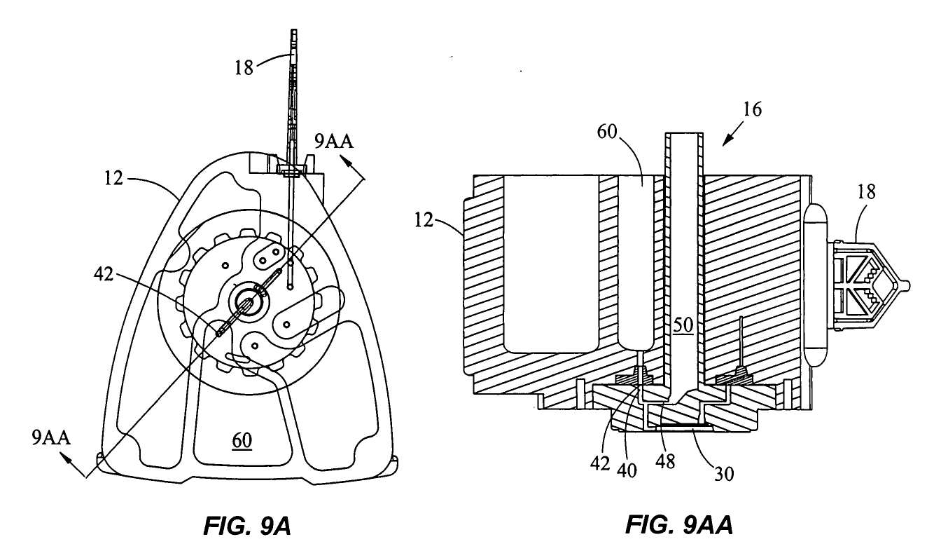

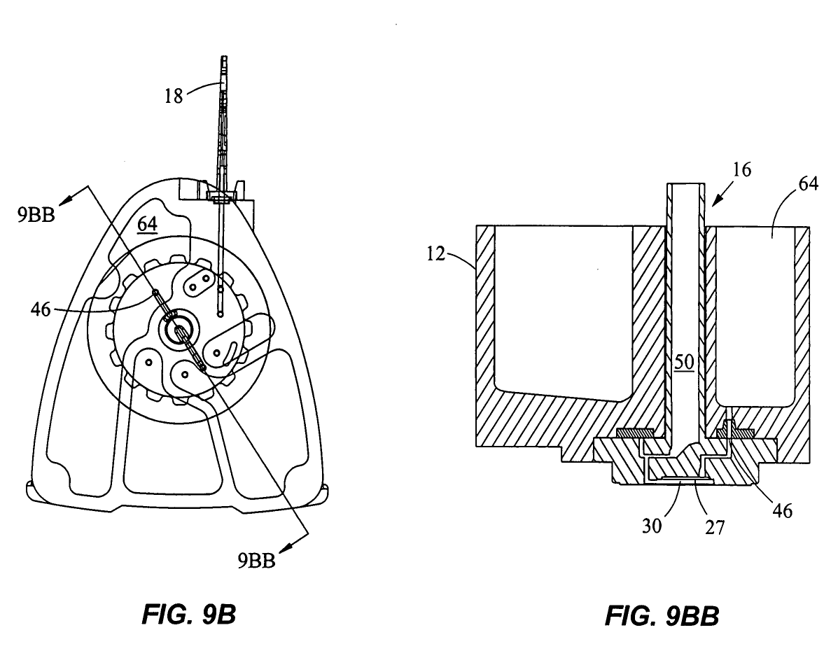

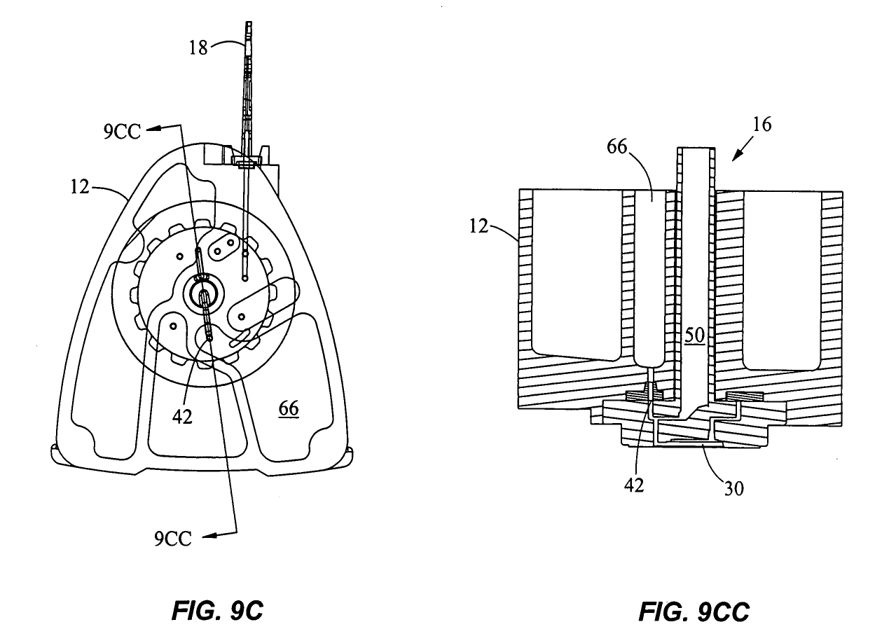

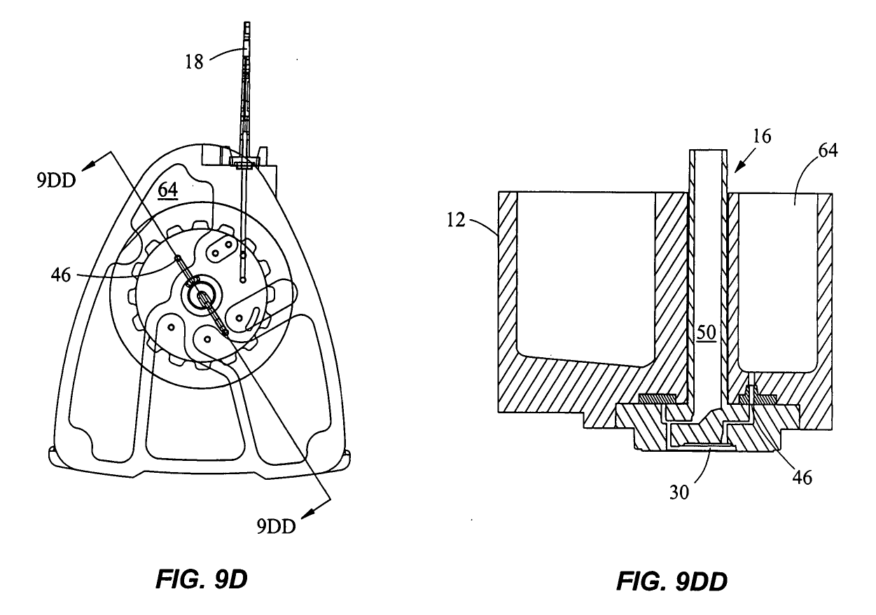

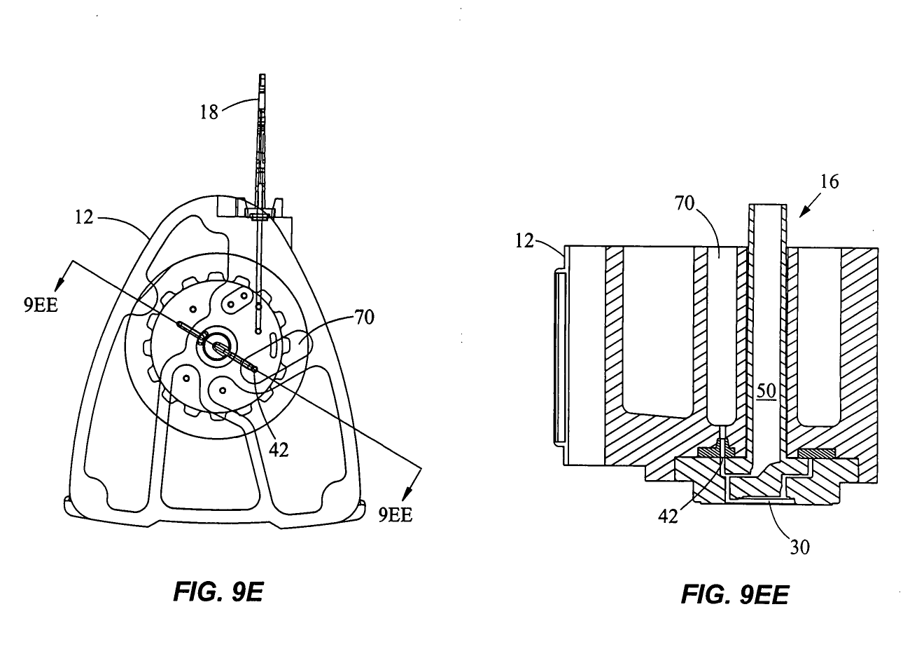

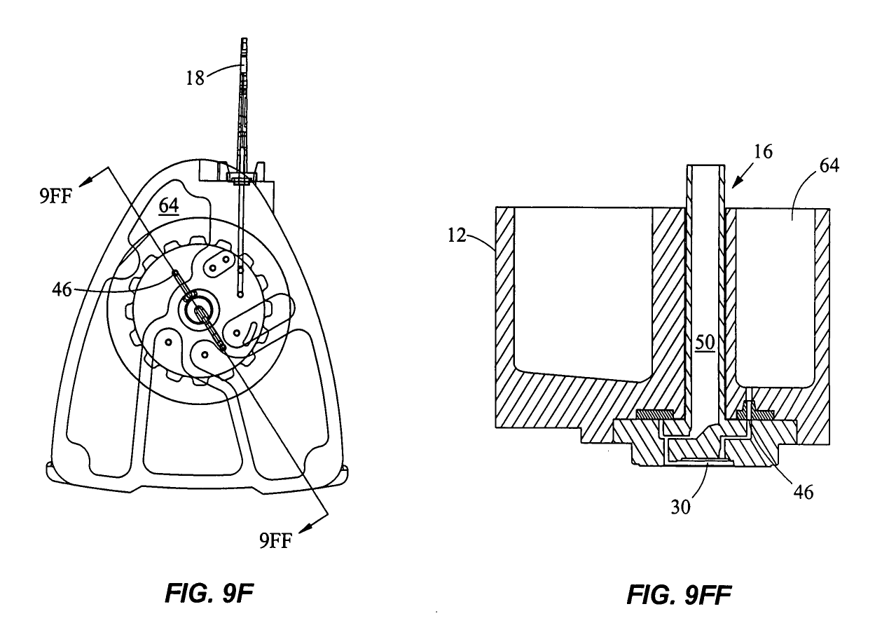

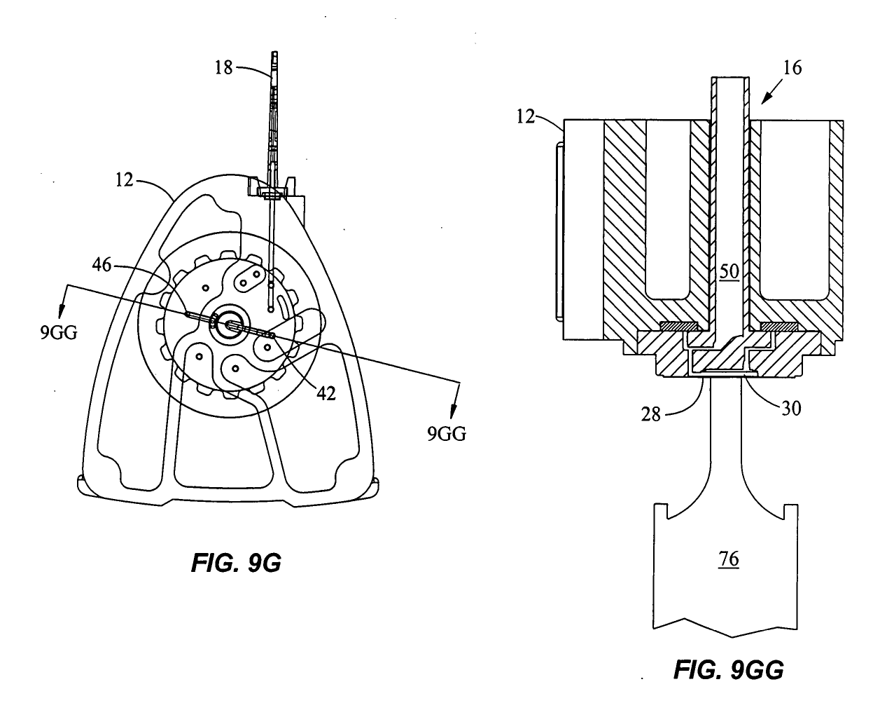

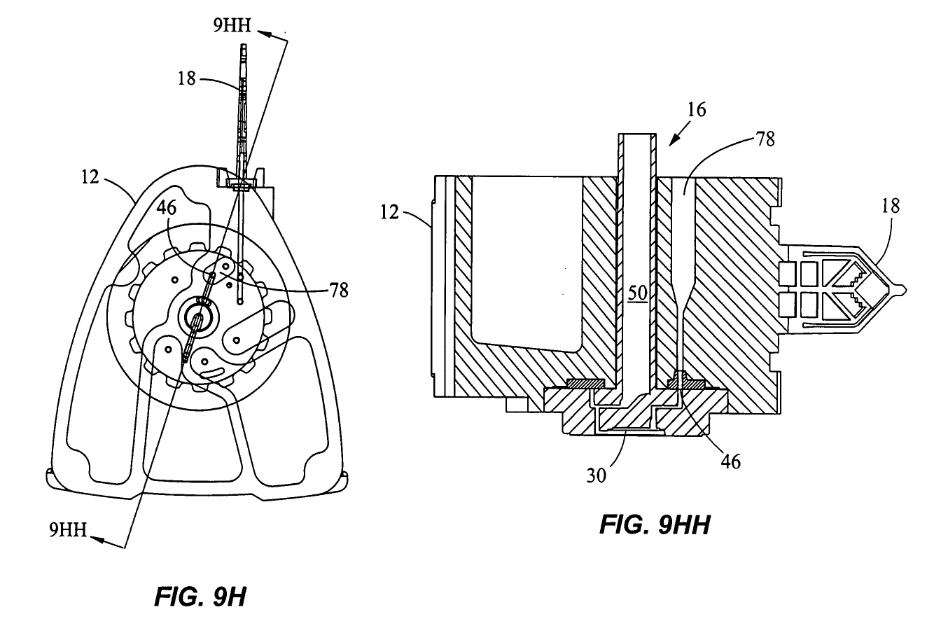

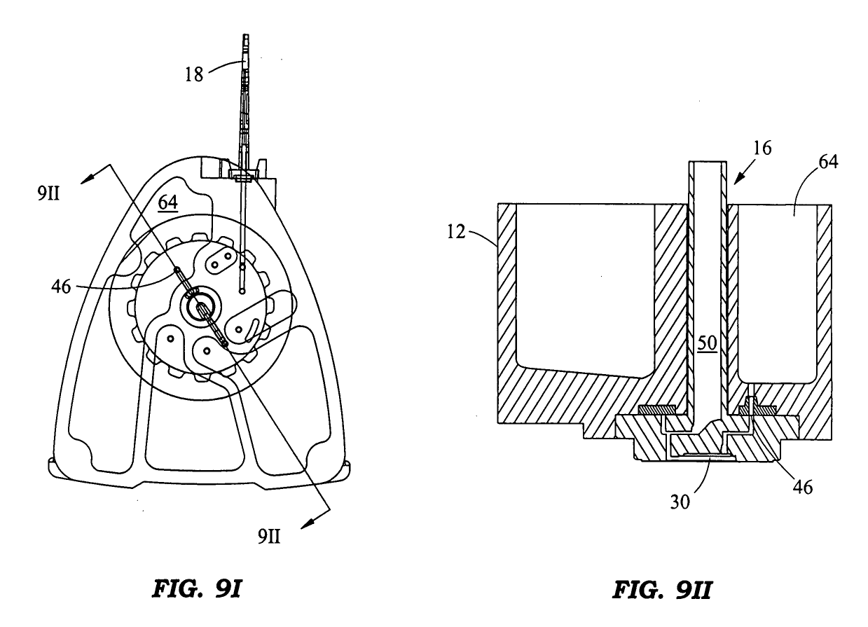

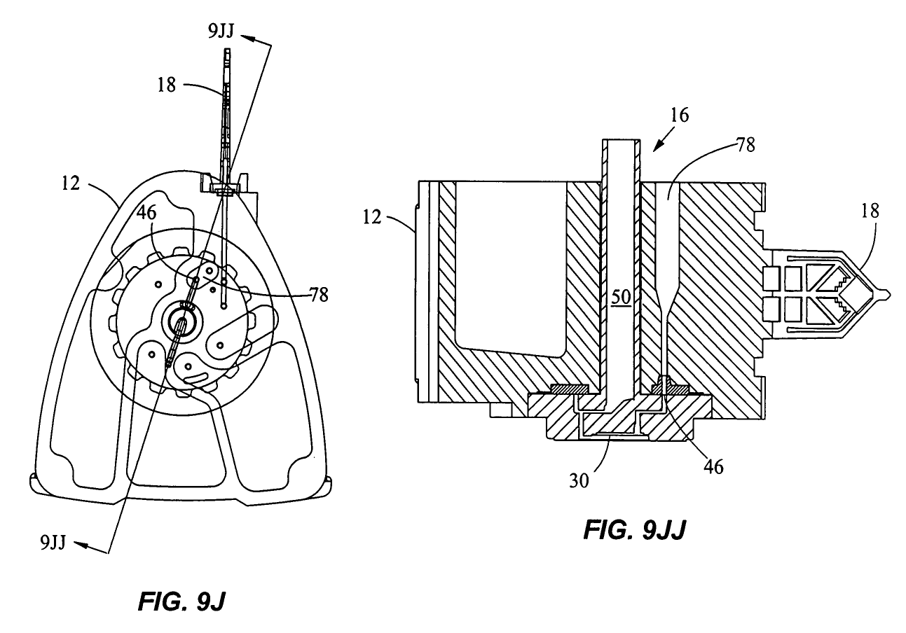

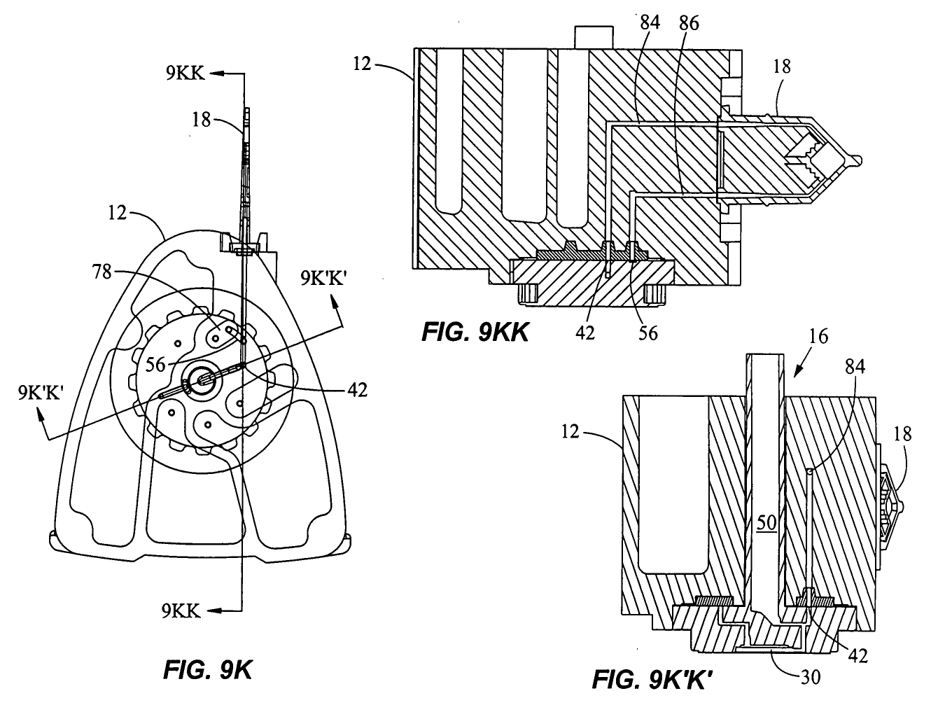

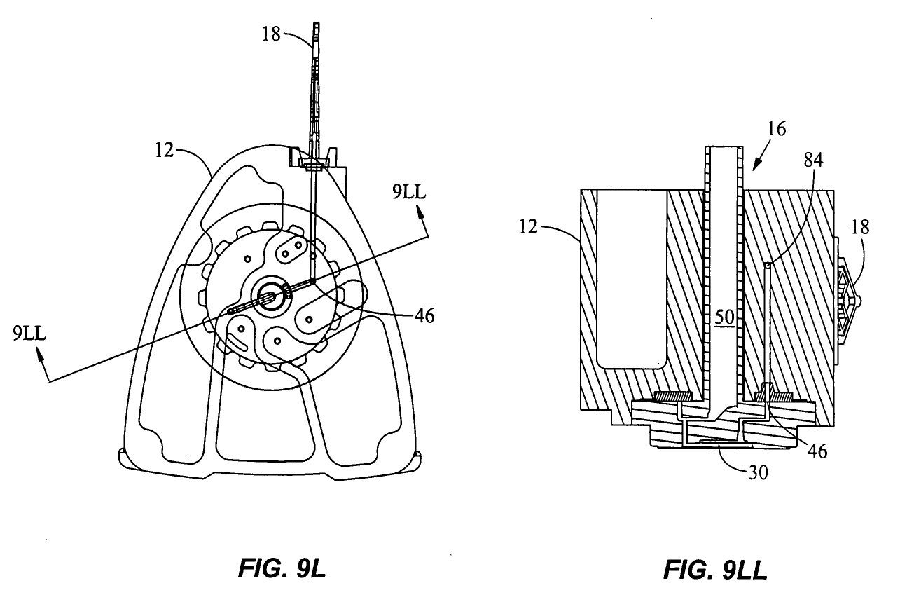

FIGS. 9A-9LL illustrate the operation of the valve 16 for conducting a nucleic acid assay of a sample suspected of containing one or more target entities (e.g., cells, spores, viruses, or microorganisms). The target entities comprise at least one target nucleic acid sequence for which the sample is being tested. A sample may be introduced into the housing 12 of the fluid control and processing device 10, which may be configured as a cartridge, by a variety of mechanisms, manual or automated. For manual addition, a measured volume of material may be placed into a receiving area of the housing 12 (e.g., one of the plurality of chambers) through an input port and a cap is then placed over the port. Alternatively, the receiving area may be covered by a rubber or similar barrier and the sample is injected into the receiving area by puncturing the barrier with a needle and injecting the sample through the needle. Alternatively, a greater amount of sample material than required for the analysis can be added to the housing 12 and mechanisms within the housing 12 can effect the precise measuring and aliquoting of the sample needed for the specified protocol.

It may be desirable to place certain samples, such as tissue biopsy material, soil, feces, exudates, and other complex material into another device or accessory and then place the secondary device or accessory into the housing causing a mechanical action which effects a function such as mixing, dividing, or extraction. For example, a piece of tissue may be placed into the lumen of a secondary device that serves as the input port cap. When the cap is pressed into the port, the tissue is forced through a mesh that slices or otherwise divides the tissue.

For automated sample introduction, additional housing or cartridge design features are employed and, in many cases, impart sample collection functionality directly into the housing. With certain samples, such as those presenting a risk of hazard to the operator or the environment, such as human retrovirus pathogens, the transfer of the sample to the housing may pose a risk. Thus, in one embodiment, a syringe or sipper may be integrated into the device to provide a means for moving a sample directly into the housing. Alternatively, the device may include a venous puncture needle and a tube forming an assembly that can be used to acquire a sample. After collection, the tube and needle are removed and discarded, and the housing 12 is then placed in an instrument to effect processing. The advantage of such an approach is that the operator or the environment is not exposed to pathogens.

The input port can be designed with a consideration of appropriate human factors as a function of the nature of the intended specimen. For example, respiratory specimens may be acquired from the lower respiratory tract as expectorants from coughing. Swab or brush samples may also be placed into the device. In the former case, the input port can be designed to allow the patient to cough directly into the housing 12 or to otherwise facilitate spitting of the expectorated sample into the housing. For brush or swab samples, the brush or swab is preferably placed in one of the chambers of the device 10 and the sample is eluted off the brush or swab using, e.g., water or other suitable elution fluid. In addition, the housing 12 may include features that facilitate the breaking off and retaining of the end of the swab or brush in the sample-receiving chamber.

In another embodiment, the housing 12 includes one or more input tubes or sippers that may be positioned in a sample pool so that the sample material flows into the housing 12. Alternatively, a hydrophilic wicking material can function to draw a sample into the device. For example, the entire cartridge can be immersed directly into the sample, and a sufficient amount of sample is absorbed into the wicking material and wicks into the housing 12. The housing is then removed, and can be transported to the laboratory or analyzed directly using a portable instrument. In another embodiment, tubing can be utilized so that one end of the tube is in direct communication with the housing to provide a fluidic interface with at least one chamber and the other end is accessible to the external environment to serve as a receiver for sample. The tube can then be placed into a sample and serve as a sipper. Thus, the device may include a variety of features for collecting a sample from various different sources and for moving the sample into the housing 12, thereby reducing handling and inconvenience.

In FIGS. 9A and 9AA, a sample is placed in a mixing chamber 60, e.g., by pipetting, and then a lid is placed over the chamber 60. The sample will be tested to determine if it contains one or more target nucleic acid sequences. This requires sample preparation steps, e.g., lysing the target cells, spores, viruses, or microorganisms containing the target nucleic acid sequence. The chamber 60 contains sample preparation controls to be mixed with the sample. The sample preparation controls are also cells, spores, viruses, or microorganisms. The sample preparation controls contain a marker nucleic acid sequence different than the target nucleic acid sequence for which the sample is being assayed. The marker nucleic acid sequence will be detected in the reaction chamber 18 later in the assay, along with the target nucleic acid sequence if the target nucleic acid sequence is present in the sample. In order for the marker nucleic acid sequence to be detected, the sample preparation controls must be successfully lysed to release their nucleic acid and the nucleic acid must be successfully mixed with amplification reagents and amplified. The sample preparation controls thus indicate that sample preparation was adequate for the nucleic acid assay if they can be detected and inadequate if they cannot be detected. The sample preparation controls thus verify that the sample preparation was effective if they can be positively detected, so that one can feel confident in the assay results.

In one preferred embodiment, the sample preparation controls are spores containing a specific marker nucleic acid sequence to be amplified and detected. For example, 2,000 to 10,000 spores containing a specific marker nucleic acid sequence are generally preferred, and more preferably about 6,000 spores are used as the sample preparation controls. The spores should be cleaned so that there is no external nucleic acid in order to prove that lysis step of the sample preparation is effective, and not just loosening external nucleic acid. In addition, the sample preparation controls are preferably stored in one of the chambers of the housing 12 in a lyophilized or dried-down bead that is quickly dissolvable in liquid. Methods for making such beads are well known in the art and are described in U.S. Pat. No. 5,593,824 and in co-pending U.S. patent application Ser. No. 10/672,266 filed Sep. 25, 2003, the disclosures of which are incorporated by reference herein.

The sample suspected of containing target cells, spores, viruses, or microorganisms is mixed with the sample preparation controls in the chamber 60. The mixing is preferably accomplished by dissolving a dried bead containing the sample preparation controls in the sample fluid. The first external port 42 is placed in fluidic communication with the chamber 60 by rotating the valve 16, and the piston 54 is pulled upward to draw a fluid sample from the chamber 60 through the first outer conduit 40 and fluid displacement channel 48 to the fluid displacement chamber 50, bypassing the lysing chamber 30. For simplicity, the piston 54 is not shown in FIGS. 9A-9LL. The valve 16 is then rotated to place the second external port 46 in fluidic communication with a waste chamber 64 as shown in FIGS. 9B and 9BB. The piston 54 is pushed downward to drive the fluid sample mixed with the sample preparation controls through the lysing chamber 30 to the waste chamber 64. In a specific embodiment, the lysing chamber 30 includes at least one filter 27 having a pore size sufficient for capturing the target cells, spores, viruses, or microorganisms, if present in the sample, as well as capturing the sample preparation controls, as the sample fluid passes through the lysing chamber 30. For this reason, it is desirable that the sample preparation controls have the same approximate size or be slightly smaller than the target cells, spores, viruses, or microorganisms in the sample to prove that the filtration of the target entities, if they were present in the sample, was successful. In alternative embodiments, other solid phase materials may be provided in the lysing chamber 30.

In FIGS. 9C and 9CC, the valve 16 is rotated to place the first external port 42 in fluidic communication with a wash chamber 66, and the piston 54 is pulled upward to draw a wash fluid from the wash chamber 66 into the fluid displacement chamber 50, bypassing the lysing chamber 30. The valve 16 is then rotated to place the second external port 46 in fluidic communication with the waste chamber 64 as shown in FIGS. 9D and 9DD. The piston 54 is pushed downward to drive the wash fluid through the lysing chamber 30 to the waste chamber 64. The above washing steps may be repeated as desired. The intermediate washing is used to remove unwanted residue within the valve 16.

In FIGS. 9E and 9EE, the valve 16 is rotated to place the first external port 42 in fluidic communication with a buffer chamber 70, and the piston 54 is pulled upward to draw a lysis buffer (e.g., water or water mixed with lysing agents) from the buffer chamber 70 into the fluid displacement chamber 50, bypassing the lysing chamber 30. The valve 16 is then rotated to place the second external port 46 in fluidic communication with the waste chamber 64 as shown in FIGS. 9F and 9FF. The piston 54 is pushed downward to drive the buffer fluid into the lysing chamber 30. In FIGS. 9G, and 9GG, the valve 16 is rotated to close the external ports 42, 46.

The sample preparation controls and the target cells, viruses, spores, or microorganisms, if present, are subjected to a lysis treatment in the lysing chamber 30. The purpose of the lysis treatment is to break the outer walls of the sample preparation controls and of the target cells, viruses, spores, or microorganisms, if present, to release their nucleic acid. The sample preparation controls are preferably the same level of difficulty or more difficult to lyse than the target cells, viruses, spores, or microorganisms to prove that the lysis treatment was effective. Liberation of nucleic acids from the cells, viruses, spores, or microorganisms, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Where chemical extraction and/or denaturation methods are used, the appropriate lysing agents are preferably in the lysis buffer stored in the chamber 70 and pumped into the lysing chamber 30.

Alternatively, physical methods may be used to extract the nucleic acids and denature DNA binding proteins. U.S. Pat. No. 5,304,487, incorporated herein by reference in its entirety for all purposes, discusses the use of physical protrusions within microchannels or sharp edged particles within a chamber or channel to pierce cell membranes and extract their contents. Combinations of such structures with piezoelectric elements for agitation can provide suitable shear forces for lysis. More traditional methods of cell extraction may also be used, e.g., employing a channel with restricted cross-sectional dimension which causes cell lysis when the sample is passed through the channel with sufficient flow pressure. Alternatively, cell extraction and denaturing of contaminating proteins may be carried out by applying an alternating electrical current. A variety of other methods may be utilized within the device of the present invention to effect cell lysis/extraction, including, e.g., subjecting cells to ultrasonic agitation, or forcing cells through microgeometry apertures, thereby subjecting the cells to high shear stress resulting in rupture.

In one preferred embodiment, the lysis treatment comprises sonicating the lysing chamber 30 using an ultrasonic transducer 76 coupled to the outer wall 28 of the lysing chamber 30. The ultrasonic transducer 76, preferably an ultrasonic horn, is placed in contact with the wall 28 to transmit ultrasonic energy into the lysing chamber 30 to facilitate lysing of the cells, spores, viruses, or microorganisms. Suitable ultrasonic horns are commercially available from Sonics & Materials, Inc. having an office at 53 Church Hill, Newton, Connecticut 06470-1614, U.S.A. Alternatively, the ultrasonic transducer may comprise a piezoelectric disk or any other type of ultrasonic transducer that may be coupled to the wall 28. In addition, beads (e.g., glass or polystyrene beads) are preferably agitated in the lysing chamber 30 to rupture the cells, spores, viruses, or microorganisms. The pressure waves or pressure pulses created by the transducer 76 vibrating against the wall 28 causes the beads to move in ballistic motion in the lysis buffer and cause the rupturing. In these embodiments employing an ultrasonic transducer, the lysis buffer should be an ultrasonic transmission medium, e.g., deionized water. The lysis buffer may also include one or more lysing agents to aid in the lysis. In the presently preferred embodiment, the wall 28 is a deflectable plastic wall as described in co-pending U.S. patent application Ser. No. 09/972,221 filed Oct. 4, 2001 the disclosure of which is incorporated by reference herein.

In FIGS. 9H and 9HH, the valve 16 is rotated to place the second external port 46 in fluidic communication with a reagent chamber 78, and the piston 54 is pushed downward to elute the lysate in the lysing chamber 30 to the reagent chamber 78. The reagent chamber 78 preferably contains all of the necessary nucleic acid amplification reagents and probes (e.g., enzyme, primers, and fluorescent probes) to amplify and detect the marker nucleic acid sequence of the sample preparation controls and the one or more target nucleic acid sequences for which the sample is being tested. Any excess lysate is dispensed into the waste chamber 64 via the second external port 46 after rotating the valve 16 to place the port 46 in fluidic communication with the waste chamber 64, as shown in FIGS. 9I and 9II. The lysate containing nucleic acid released in the lysing chamber 30 is then mixed in the reagent chamber 78. This is carried out by placing the fluid displacement chamber 50 in fluidic communication with the reagent chamber 78 as shown in FIGS. 9J and 9JJ, and moving the piston 54 up and down. Toggling of the mixture through the filter in the processing region 30, for instance, allows larger particles trapped in the filter to temporarily move out of the way to permit smaller particles to pass through.

The reagents and probes for amplifying and detecting the marker nucleic acid sequence of the sample preparation controls and the one or more target nucleic acid sequences for which the sample is being tested are preferably stored in chamber 78 in a lyophilized or dried-down bead that is quickly dissolvable in liquid. Methods for making such beads are well known in the art and are described in U.S. Pat. No. 5,593,824 and in co-pending U.S. patent application Ser. No. 10/672,266 filed Sep. 25, 2003, the disclosures of which are incorporated by reference herein. In an alternative embodiment, the reagents and probes are stored in the reaction chamber of the reaction vessel 18.

In FIGS. 9K, 9KK, and 9K′K′, the valve 16 is rotated to place the first external port 42 in fluidic communication with a first branch 84 coupled to the reaction vessel 18, while the second branch 86 which is coupled to the reaction vessel 18 is placed in fluidic communication with the crossover groove 56. The first branch 84 and second branch 86 are disposed at different radii from the axis 52 of the valve 16, with the first branch 84 having a common radius with the first external port 42 and the second branch 86 having a common radius with the crossover groove 56. The crossover groove 56 is also in fluidic communication with the reagent chamber 78 (FIG. 9K), and serves to bridge the gap between the reagent chamber 78 and the second branch 86 to provide crossover flow therebetween. The external ports are disposed within a range of external port radii from the axis and the crossover groove is disposed within a range of crossover groove radii from the axis, where the range of external port radii and the range of crossover groove radii are non-overlapping. Placing the crossover groove 56 at a different radius from the radius of the external ports 42, 46 is advantageous because it avoids cross-contamination of the crossover groove 56 by contaminants that may be present in the area near the surfaces between the valve 16 and the housing 12 at the radius of the external ports 42, 46 as a result of rotational movement of the valve 16. Thus, while other configurations of the crossover groove may be used including those that overlap with the radius of the external ports 42, 46, the embodiment as shown is a preferred arrangement that isolates the crossover groove 56 from contamination from the area near the surfaces between the valve 16 and the housing 12 at the radius of the external ports 42, 46.

To fill the reaction vessel 18, the piston 54 is pulled upward to draw the reaction mixture in the reagent chamber 78 through the crossover groove 56 and the second branch 86 into the reaction vessel 18. The valve 16 is then rotated to place the second external port 46 in fluidic communication with the first branch 84 and to close the first external port 42, as shown in FIGS. 9L and 9LL. The piston 54 is pushed downward to pressurize the reaction mixture inside the reaction vessel 18. The reaction vessel 18 has a reaction chamber for holding the reaction mixture for nucleic acid amplification and detection. The reaction chamber may be inserted into a thermal reaction sleeve for performing nucleic acid amplification and detection. The two branches 84, 86 allow filling and evacuation of the reaction chamber of the reaction vessel 18. The vessel maybe connected to the housing 12 by ultrasonic welding, mechanical coupling, or the like, or be integrally formed with the housing 12 such as by molding.

The reaction mixture in the reaction chamber of the vessel 18 is subjected to nucleic acid amplification conditions. Amplification of an RNA or DNA template using reactions is well known (see U.S. Pat. Nos. 4,683,195 and 4,683,202; PCR Protocols: A Guide to Methods and Applications (Innis et al., eds, 1990)). Methods for amplifying and detecting nucleic acids by PCR using a thermostable enzyme are disclosed in U.S. Pat. No. 4,965,188, which is incorporated herein by reference. PCR amplification of DNA involves repeated cycles of heat-denaturing the DNA, annealing two oligonucleotide primers to sequences that flank the DNA segment to be amplified, and extending the annealed primers with DNA polymerase. The primers hybridize to opposite strands of the target sequence and are oriented so that DNA synthesis by the polymerase proceeds across the region between the primers, effectively doubling the amount of the DNA segment. Moreover, because the extension products are also complementary to and capable of binding primers, each successive cycle essentially doubles the amount of DNA synthesized in the previous cycle. This results in the exponential accumulation of the specific target fragment, at a rate of approximately 2n per cycle, where n is the number of cycles. Methods such as polymerase chain reaction (PCR) and ligase chain reaction (LCR) can be used to amplify nucleic acid sequences of target DNA sequences directly from mRNA, from cDNA, from genomic libraries or cDNA libraries.

Isothermic amplification reactions are also known and can be used according to the methods of the invention. Examples of isothermic amplification reactions include strand displacement amplification (SDA) (Walker, et al. Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313):91-2 (1991), rolling circle amplification (RCA) (Lisby, Mol. Biotechnol. 12(1):75-99 (1999)); Hatch et al., Genet. Anal. 15(2):35-40 (1999)) and branched DNA signal amplification (bDNA) (see, e.g., Iqbal et al., Mol. Cell Probes 13(4):315-320 (1999)). Other amplification methods known to those of skill in the art include CPR (Cycling Probe Reaction), SSR (Self-Sustained Sequence Replication), SDA (Strand Displacement Amplification), QBR (Q-Beta Replicase), Re-AMP (formerly RAMP), RCR (Repair Chain Reaction), TAS (Transcription Based Amplification System), and HCS.

The nucleic acid amplification reaction is preferably carried out using a thermal processing instrument that heats and/or cools the reaction mixture in the vessel 18 to the temperatures needed for the amplification reaction. The thermal processing instrument can also optionally comprise one or more detection mechanisms for detecting the marker nucleic acid sequence of the sample preparation controls and the one or more target nucleic acid sequences for which the sample is being tested. A preferred thermal processing instrument with built in optical detectors for amplifying and detecting nucleic acid sequences in the vessel 18 is described in commonly assigned U.S. Pat. Nos. 6,369,893 and 6,391,541, the disclosures of which are incorporated by reference herein. There are also many other known ways to control the temperature of a reaction mixture and detect nucleic acid sequences in the reaction mixture that are suitable for the present invention. For example, other instruments for nucleic acid amplification and detection are described, e.g., in U.S. Pat. Nos. 5,958,349; 5,656,493; 5,333,675; 5,455,175; 5,589,136 and 5,935,522.

The detection of the marker nucleic acid sequence of the sample preparation controls and of the one or more target nucleic acid sequences for which the sample is being tested is preferably carried out using probes. The reaction vessel 18 preferably has one or more transparent or light-transmissive walls through which signals from the probe may be optically detected. Preferably hybridization probes are used to detect and quantify the nucleic acid sequences. There are many different types of assays that employ nucleic acid hybridization probes. Some of these probes generate signals with a change in the fluorescence of a fluorophore due to a change in its interaction with another molecule or moiety. Typically, the interaction is brought about by changing the distance between the fluorophore and the interacting molecule or moiety. These assays rely for signal generation on fluorescence resonance energy transfer, or “FRET.” FRET utilizes a change in fluorescence caused by a change in the distance separating a first fluorophore from an interacting resonance energy acceptor, either another fluorophore or a quencher. Combinations of a fluorophore and an interacting molecule or moiety, including quenching molecules or moieties, are known as “FRET pairs.” The mechanism of FRET-pair interaction requires that the absorption spectrum of one member of the pair overlaps the emission spectrum of the other member, the first fluorophore. If the interacting molecule or moiety is a quencher, its absorption spectrum must overlap the emission spectrum of the fluorophore. Stryer, L., Ann. Rev. Biochem. 1978, 47: 819-846; BIOPHYSICAL CHEMISTRY part II, Techniques for the Study of Biological Structure and Function, (C. R. Cantor and P. R. Schimmel, eds., 1980), pages 448-455, and Selvin, P. R., Methods in Enzymology 246: 300-335 (1995). Efficient, or a substantial degree of, FRET interaction requires that the absorption and emission spectra of the pair have a large degree of overlap. The efficiency of FRET interaction is linearly proportional to that overlap. Haugland, R. P., Yguerabide, Jr., and Stryer, L., Proc. Natl. Acad. Sci. USA 63: 24-30 (1969). Non-FRET probes have also been described. See, e.g., U.S. Pat. No. 6,150,097.

Another preferred method for detection of amplification products is the 5′ nuclease PCR assay (also referred to as the TaqMan® assay) (Holland et al., Proc. Natl. Acad. Sci. USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the “TaqMan®” probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

Another method of detecting amplification products that relies on the use of energy transfer is the “beacon probe” method described by Tyagi and Kramer (Nature Biotech. 14:303-309 (1996)), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus when the beacon is in the open conformation, the fluorescence of the donor fluorophore is detectable, whereas when the beacon is in hairpin (closed) conformation, the fluorescence of the donor fluorophore is quenched. When employed in PCR, the molecular beacon probe, which hybridizes to one of the strands of the PCR product, is in “open conformation,” and fluorescence is detected, while those that remain unhybridized will not fluoresce (Tyagi and Kramer, Nature Biotechnol. 14: 303-306 (1996). As a result, the amount of fluorescence will increase as the amount of PCR product increases, and thus may be used as a measure of the progress of the PCR.

To be confident about the detection, or lack thereof, of a target nucleic acid sequence in a sample, one should control for the integrity of the sample preparation.

This is why the sample preparation controls are subjected to the same treatment as the target entities (e.g., target cells, spores, viruses, or microorganisms containing a target nucleic acid sequence) in the sample. If the marker nucleic acid sequence of the sample preparation controls is detected, then the sample preparation is deemed satisfactory. If the presence of the marker nucleic acid sequence cannot be detected, then the sample preparation is deemed inadequate and the outcome of the test for the target nucleic acid sequence is deemed “unresolved”. Preferably, the presence or absence of the marker nucleic acid sequence is detected by determining if a signal from a hybridization probe capable of binding to the marker nucleic acid sequence exceeds a threshold level, e.g., a predetermined fluorescent threshold level that must be met or exceeded for the assay to be deemed valid.

To operate the valve 16 of FIGS. 3-8, a motor such as a stepper motor is typically coupled to the toothed periphery 29 of the disk portion 22 to rotate the valve 16 relative to the housing 12 for distributing fluid with high precision. The motor can be computer-controlled according to the desired protocol. A linear motor or the like is typically used to drive the piston 54 up and down with precision to provide accurate metering, and may also be computer-controlled according to the desired protocol.

The use of a single valve produces high manufacturing yields due to the presence of only one failure element. The concentration of the fluid control and processing components results in a compact apparatus (e.g., in the form of a small cartridge) and facilitates automated molding and assembly. As discussed above, the system advantageously includes dilution and mixing capability, intermediate wash capability, and positive pressurization capability. The fluid paths inside the system are normally closed to minimize contamination and facilitate containment and control of fluids within the system. The reaction vessel is conveniently detachable and replaceable, and may be disposable in some embodiments.

The components of the fluid control and processing system may be made of a variety of materials that are compatible with the fluids being used. Examples of suitable materials include polymeric materials such as polypropylene, polyethylene, polycarbonate, acrylic, or nylon. The various chambers, channels, ports, and the like in the system may have various shapes and sizes.

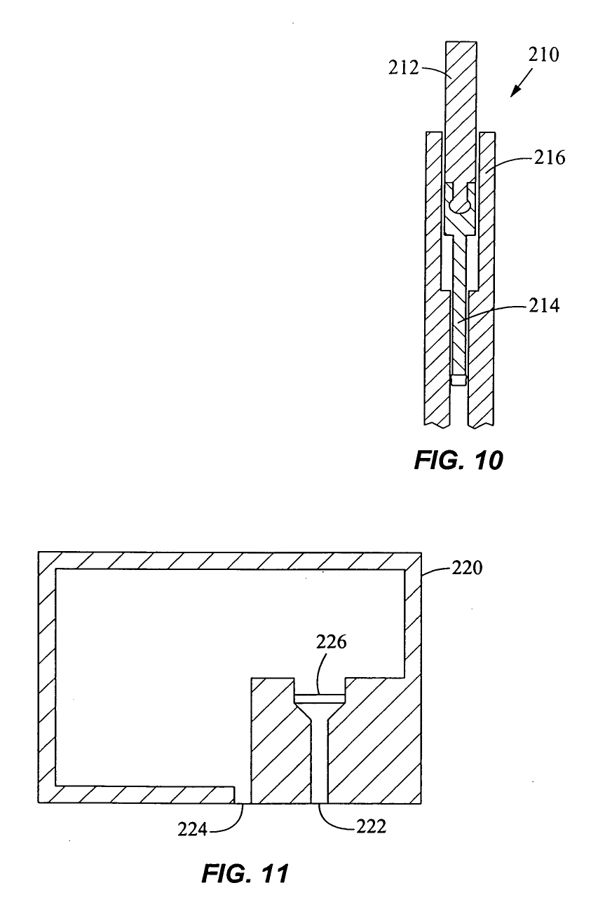

FIG. 10 shows another embodiment in which a piston assembly 210 including a piston rod 212 connected to a piston shaft 214 having a smaller cross-section than the rod 212 for driving small amounts of fluids. The thin piston shaft 214 may bend under an applied force if it is too long. The piston rod 212 moves along the upper portion of the barrel or housing 216, while the piston shaft 214 moves along the lower portion of the barrel 216. The movement of the piston rod 212 guides the movement of the piston shaft 214, and absorbs much of the applied force so that very little bending force is transmitted to the thin piston shaft 214.

FIG. 11 shows another embodiment in which the sample is pre-filtered before being mixed with the sample preparation controls. The sample is preferably pre-filtered in a side chamber 220 that is incorporated into the device. The side chamber 220 includes an inlet port 222 and an outlet port 224. In this example, the side chamber 220 includes a filter 226 disposed at the inlet port 222. Sample fluid is directed to flow via the inlet port 222 into the side chamber 220 and out via the outlet port 224 for side filtering. This allows filtering of a fluid sample or the like using the fluid control device of the invention. The fluid may be recirculated to achieve better filtering by the filter 226. This prefiltering is useful to remove coarse material, that might otherwise clog up the other parts of the device, before mixing the sample with the sample preparation controls. After the sample is pre-filtered, it is mixed with the sample preparation controls, e.g., in the chamber 66 of FIG. 9C or another chamber of the housing 12. The use of a side chamber is advantageous, for instance, to avoid contaminating the valve and the other chambers in the device.

The above-described arrangements of devices and methods are merely illustrative of applications of the principles of this invention and many other embodiments and modifications may be made without departing from the spirit and scope of the invention as defined in the claims.

For example, although a rotary-valve cartridge has been described as a preferred embodiment, the sample preparation control of the present invention is suitable for many other cartridge designs. Alternative cartridge designs are described in U.S. Pat. Nos. 6,391,541, 6,440,725, and 6,168,948 the disclosures of which are incorporated by reference herein. Moreover, when a rotary valve cartridge is used, the cartridge may have more or fewer chamber than shown in the preferred embodiments and many different sample preparation protocols may be executed.

The scope of the invention should, therefore, be determined not with reference to the above description, but instead should be determined with reference to the appended claims along with their full scope of equivalents.

1. A method for preparing a sample for a nucleic acid amplification reaction and for verifying the effectiveness of the sample preparation, the sample being suspected of containing target entities selected from the group consisting of cells, spores, microorganisms, and viruses, the target entities comprising at least one target nucleic acid sequence, the method comprising the steps of:
a) introducing the sample into a device having:
i) a mixing chamber for mixing the sample with sample preparation controls, the sample preparation controls being selected from the group consisting of cells, spores, microorganisms, and viruses, and the sample preparation controls comprising a marker nucleic acid sequence;
ii) a lysing chamber; and
iii) a reaction chamber;
b) mixing the sample with the sample preparation controls in the mixing chamber;
c) subjecting the sample preparation controls and the target entities, if present in the sample, to a lysis treatment in the lysing chamber;
d) subjecting nucleic acid released in the lysing chamber to nucleic acid amplification conditions in the reaction chamber; and
e) detecting the presence or absence of the target nucleic acid sequence and of the marker nucleic acid sequence;
whereby detection of the marker nucleic acid sequence indicates satisfactory sample preparation.

2. The method of claim 1, wherein the lysing chamber contains solid phase material, and the method further comprises the step of forcing the sample mixed with the sample preparation controls to flow through the lysing chamber to capture the sample preparation controls and the target entities, if present in the sample, with the solid phase material prior to the lysis treatment.

3. The method of claim 2, wherein the solid phase material comprises at least one filter having a pore size sufficient to capture the sample preparation controls and the target entities.

4. The method of claim 3, further comprising the step of pre-filtering the sample prior to mixing the sample with the sample preparation controls.

5. The method of claim 3, wherein the lysis treatment comprises subjecting the sample preparation controls and the target entities to ultrasonic energy using an ultrasonic transducer coupled to a wall of the lysing chamber.

6. The method of claim 5, wherein the lysis treatment further comprises agitating beads in the lysing chamber.

7. The method of claim 1, wherein the sample preparation controls are spores.

8. The method of claim 1, wherein the mixing step comprises dissolving a dried bead containing the sample preparation controls.

9. The method of claim 1, wherein the lysis treatment comprises subjecting the sample preparation controls and the target entities to ultrasonic energy using an ultrasonic transducer coupled to a wall of the lysing chamber.

10. The method of claim 9, wherein the lysis treatment further comprises agitating beads in the lysing chamber to rupture the sample preparation controls and the target entities.

11. The method of claim 1, wherein the lysis treatment comprises contact with a chemical lysis agent.

12. The method of claim 1, wherein the nucleic acid amplification conditions comprise polymerase chain reaction (PCR) conditions.

13. The method of claim 1, wherein the presence or absence of the marker nucleic acid sequence is detected by determining if a signal from a probe capable of binding to the marker nucleic acid sequence exceeds a threshold level.

14. A device for preparing a sample for a nucleic acid amplification reaction and for verifying the effectiveness of the sample preparation, the sample being suspected of containing target entities selected from the group consisting of cells, spores, microorganisms, and viruses, the target entities comprising at least one target nucleic acid sequence, the device comprising a body having:
a) a first chamber containing sample preparation controls to be mixed with the sample, the sample preparation controls being selected from the group consisting of cells, spores, microorganisms, and viruses, and the sample preparation controls comprising a marker nucleic acid sequence;
b) a lysing chamber for subjecting the sample preparation controls and the target entities, if present in the sample, to a lysis treatment to release the nucleic acid therefrom;
c) a reaction chamber for holding the nucleic acid for amplification and detection; and
d) at least one flow controller for directing the sample mixed with the sample preparation controls to flow from the first chamber into the lysing chamber and for directing the nucleic acid released in the lysing chamber to flow into the reaction chamber, wherein the device further contains primers and probes for amplifying and detecting the marker nucleic acid sequence and the at least one target nucleic acid sequence.

15. The device of claim 14, wherein the lysing chamber contains solid phase material for capturing the sample preparation controls and the target entities, if present in the sample, as the sample flows through the lysing chamber, the device further includes at least one waste chamber for receiving used sample fluid that has flowed through the lysing chamber, and the at least one flow controller is further capable of directing used sample fluid that has flowed through the lysing chamber to flow into the waste chamber.

16. The device of claim 15, wherein the solid phase material comprises at least one filter having a pore size sufficient to capture the sample preparation controls and the target entities.

17. The device of claim 16, further comprising an ultrasonic transducer coupled to a wall of the lysing chamber to sonicate the lysing chamber.

18. The device of claim 17, further comprising beads in the lysing chamber for rupturing the sample preparation controls and the target entities.

19. The device of claim 14, wherein the sample preparation controls are spores.

20. The device of claim 14, wherein the sample preparation controls are in a dried bead that is dissolvable in liquid.

21. The device of claim 14, wherein the primers and probes are in a dried bead in the reaction chamber, the bead being dissolvable in liquid.

22. The device of claim 14, wherein the body includes a reagent chamber connected to the reaction chamber, and wherein the primers and probes are in a dried bead in the mixing chamber, the bead being dissolvable in liquid.

23. The device of claim 14, further comprising an ultrasonic transducer coupled to a wall of the lysing chamber to sonicate the lysing chamber.

24. The device of claim 23, further comprising beads in the lysing chamber for rupturing the sample preparation controls and the target entities.

25. A method for determining the effectiveness of a lysis procedure, the method comprising the steps of:
a) mixing sample preparation controls with a sample suspected of containing target entities selected from the group consisting of cells, spores, microorganisms, and viruses, wherein the target entities comprise at least one target nucleic acid sequence, and wherein the sample preparation controls are selected from the group consisting of cells, spores, microorganisms, and viruses, the sample preparation controls comprising a marker nucleic acid sequence;
b) subjecting the mixture of the sample preparation controls and the target entities, if present in the sample, to a lysis treatment;
c) detecting the presence or absence of the marker nucleic acid sequence to determine if nucleic acid was released from the sample preparation controls during the lysis treatment; whereby positive detection of the marker nucleic acid sequence indicates satisfactory lysis.

26. The method of claim 25, further comprising the step of forcing the sample mixed with the sample preparation controls to flow through a chamber containing solid phase material to capture the sample preparation controls and the target entities, if present in the sample, with the solid phase material prior to the lysis treatment.

27. The method of claim 26, wherein the solid phase material comprises at least one filter having a pore size sufficient to capture the sample preparation controls and the target entities.

28. The method of claim 27, further comprising the step of pre-filtering the sample prior to mixing the sample with the sample preparation controls.

29. The method of claim 25, wherein the lysis treatment comprises subjecting the sample preparation controls and the target entities to ultrasonic energy.

30. The method of claim 29, wherein the lysis treatment further comprises agitating beads to rupture the sample preparation controls and the target entities.

31. The method of claim 25, wherein the sample preparation controls are spores.

32. The method of claim 25, wherein the mixing step comprises dissolving a dried bead containing the sample preparation controls.

33. The method of claim 25, wherein the lysis treatment comprises contact with a chemical lysis agent.

34. The method of claim 25, wherein the marker nucleic acid sequence is detected by amplifying the marker nucleic acid sequence and detecting the amplified marker nucleic acid sequence.

35. The method of claim 34, wherein the marker nucleic acid sequence is amplified by polymerase chain reaction (PCR).

36. The method of claim 34, wherein the amplified marker nucleic acid sequence is detected by determining if a signal from a probe capable of binding to the marker nucleic acid sequence exceeds a threshold level.
